(19) United States
(12) Patent Application Publication
(10) Pub. No.: US 2017/0336422 A1
(43) Pub. Date: Nov. 23, 2017

(54) METHOD FOR THE DETECTION OF HORMONE SENSITIVE DISEASE PROGRESSION

(71) Applicant: Belgian Volition SPRL, Isnes (BE)

(72) Inventor: Jacob Vincent MICALLEF, Isnes (BE)

(73) Assignee: Belgian Volition SPRL, Isnes (BE)

(21) Appl. No.: 15/534,967

(22) PCT Filed: Dec. 10, 2015
(86) PCT No.: PCT/GB2015/053776

§ 371 Date: Jun. 9, 2017

(30) Foreign Application Priority Data
Dec. 10, 2014 (GB) 1421933.1

(51) Int. Cl. G01N 33/74; G01N 33/574

(52) U.S. Cl. CPC G01N 33/74; G01N 33/743; G01N 33/57415; G01N 33/57434; G01N 2800/52

(57) ABSTRACT

The invention relates to the use of cell free nucleosome bound nuclear hormone receptor variant adducts for detecting the progression of hormone dependent disease to hormone therapy resistant disease or the effectiveness of a drug treatment in a patient. The invention also relates to methods for detecting said cell free nucleosome bound nuclear hormone receptor variant adducts.

### FIELD OF THE INVENTION

The invention relates to a method for the detection of the progression of hormone dependent cancer disease to hormone therapy resistant disease, and to the prediction of the efficacy of drugs for patients with cancer, by measurement of circulating cell free nucleosome-nuclear hormone receptor variant adducts.

### BACKGROUND OF THE INVENTION

Prostate cancer (PCA) is a common disease in men with a high mortality rate. There is a medical need to improve the treatments available for PCA and for personalized medicine methods to predict which treatments are most appropriate to the stage of the disease, and which treatments will be most effective for individual patients. In the early stages of the disease PCA tumors need relatively high levels of androgens to grow. Such prostate cancers are often termed androgen dependent or androgen sensitive because treatments that decrease androgen levels or block androgen activity inhibit their growth. The most important androgens available to the tumor are normally circulating blood testosterone and dihydrotestosterone (DHT) produced in the testes. However, some androgens are also produced by the adrenal glands and in some cases by prostate cancer cells themselves and, whilst smaller, this effect may support the growth of some prostate cancers.

Most cases of PCA are androgen dependent on diagnosis and treatment usually includes hormone therapy to reduce the production of androgens and/or to block the activity of androgens. Examples of hormone therapy include surgical castration (orchiectomy) which reduces the testosterone levels in the blood by up to 95% and medical castration either using drugs that reduce testosterone production by the testes (for example; Luteinizing Hormone-Releasing Hormone (LHRH) agonists and LHRH antagonists). However, neither medical nor surgical castration blocks androgen production by the adrenal glands or prostate cancer cells. Other hormone therapies include drugs that do prevent adrenal and cancer cell androgen production, as well as production by the testes (for example; CYP17 inhibitors) and anti-androgen drugs which block the action of androgens by binding to the ligand binding domain of the androgen receptor. Hormone therapies may be given alone or in combinations (for example anti-androgens may be given with medical castration) as well as in combination with other non-hormonal treatments.

As the disease progresses PCA eventually becomes castration resistant (also called androgen independent/resistant or hormone independent/resistant). Disease progression may be rapid and occur over a small number of months or may be slow and occur over many years. Castration resistant tumors grow despite lack of testosterone and require other treatment. This treatment may involve surgery, cytotoxic chemotherapy, radiotherapy or other cancer treatments. However, some drugs that function through the Androgen-Androgen Receptor-Androgen Response Element axis remain effective for the treatment of castration resistant PCA in many, but not all, patients. Examples include androgen agonist drugs such as enzalutamide and CYP17 inhibitors such as abiraterone.

A major shortcoming in the current treatment of prostate cancer is that early detection of progression of hormone sensitive to castration resistant disease is difficult. This means that appropriate changes to therapy may not occur in a timely fashion leading to poor patient outcomes. Progression may be detected late when worsening symptoms occur, including for example pain, blockage of the bladder outlet or urethra. Alternatively disease progression may be detected late by direct observation of, for example; lymph node metastases or bone metastases (observed using bone scanning technologies). Increasing serum prostate specific antigen (PSA) levels may be associated with disease progression but usually painful invasive biopsy methods are used to check for disease progression. Moreover, clinicians may need to check repeatedly for disease progression during treatment to determine whether or not the therapy being administered currently is appropriate or whether the subject's treatment regime should be altered to reflect the changing nature of the tumor. Unfortunately, current tests are suboptimal and may require repeated painful biopsy on each occasion the test is performed. Moreover, such repeated testing may not be possible.

The Androgen Receptor (AR) is central to hormone therapies for PCA treatment. AR contains a DNA Binding Domain (DBD) and a Ligand Binding Domain (LBD) which binds to androgen. When androgen binding to AR occurs, a variety of effects ensue, including a migration of AR to the nucleus where it binds via the DBD to genes containing the Androgen Response Element (ARE) DNA sequence. Many genes contain the ARE sequence and AR binding regulates their expression. Binding of AR to androgen regulated genes leads to a variety of effects, which, in hormone dependent PCA, include the growth and multiplication of cancer cells. There are additionally cytoplasmic cell signaling effects of AR.

It is thought that AR, in the absence of its LBD component, is constitutively active (ie; its default position is to migrate to the nucleus, bind to ARE and exert AR effects). The function of the LBD (in the absence of any bound steroid) acts as a brake that prevents AR migration to the nucleus and binding to the ARE in the DNA. When androgen steroid binds to the LBD, this brake on AR activity is removed and the steroid bound AR migrates to the nucleus and binds to ARE, mediating androgen controlled effects.

There are a number of hypotheses on the mechanisms of castration resistant PCA, but the effectiveness of anti-androgen and CYP17 inhibitor drugs in many castration resistant PCA patients indicates that AR is still involved. Anti-androgen drugs bind avidly to the LBD of AR, without activating AR. This blocks LBD binding to androgen and prevents AR activation by androgen. CYP17 inhibitors inhibit androgen production by testicular, adrenal and prostate cancer cells, thus preventing AR activation by androgen in the circulation or by androgen produced in the prostate cancer cells themselves.

It is reported that androgen independent PCA cell lines express short truncated AR forms which lack LBD (as well as wild type AR). Short form AR is produced as splice variants of the normal protein. These short AR splice variants are androgen independent (as they have no functioning LBD) and specifically mediate ligand-independent transcriptional activation of AR target genes. It has been suggested that short form AR molecules may be involved in castration resistant PCA disease progression (Dehm et al; 2008).

More recently it has been shown that the effectiveness of anti-androgen and CYP17 inhibitor drugs in castration resistant PCA cases can be predicted by the presence of AR variant 7 (AR-V7) in the prostate cancer cells of men with the disease. However, measurement of AR-V7 in PCA cells would normally involve an invasive biopsy to obtain cells. One method to address this problem involves collection of circulating tumor cells from men and then amplifying mRNA signals associated with the splice variant AR-V7 (Antonarakis et al; 2014). However, this method has a number of limitations for routine clinical use including its complexity, high cost and the requirement for a large volume of blood (5 mls).

Breast cancer follows a similar disease progression. Early disease is hormone sensitive and the growth of estrogen dependent tumours can be slowed or prevented by therapeutic interventions aimed at prevention of estrogen binding to the estrogen receptor (ER). Examples of such treatments include the drug tamoxifen which binds to and blocks, the LBD of ER in estrogen dependent breast cancer and aromatase inhibitors which slow or prevent estrogen production. Eventually disease progression leads to hormone resistant breast cancer which will grow even in the absence of estrogen stimulation. Hormone therapies are then no longer effective and different treatments are required. The diagnosis of estrogen dependent and independent tumours is currently performed routinely by immunostaining of tumour tissue to determine the abundance of the estrogen receptor in tumour cells.

Unfortunately this test has poor clinical performance in terms of prediction of treatment efficacy and requires tissue biopsy. Clinicians may need to retest the estrogen dependency of a tumor repeatedly during the course of tumour treatment to determine whether or not further estrogen dependent treatment is appropriate or whether the subject's treatment regime should be altered to reflect the changing nature of the tumor. Unfortunately current tests are suboptimal and require repeated painful biopsy on each occasion the test is performed. Moreover, such repeated testing may not be possible.

There are two main forms of ER, known as ERα and ERβ which are different gene products encode by the ESR1 and ESR2 genes respectively. Truncated isoforms or variants of the estrogen receptor (ER) have also been reported to be present in breast cancer cells removed on biopsy from patients. Moreover these truncated ER variant forms also develop with disease progression and confer hormone therapy resistance on the tumor (Fuqua et al; 1993, Huang et al; 1997).

There are multiple variant forms of ERα. Most ERα variants expressed in breast cancer cell lines inhibit ER regulated gene expression by interfering with the ability of the wild-type ER to initiate transcription, possibly through heterodimerization with wild type ERα. In contrast, ERα variant form ERα-Δ5 activates estrogen regulated gene transcription in the absence of estrogens. ERα-Δ5 expression may therefore be a cause of hormone independent breast cancer growth and proliferation and resistance to breast cancer hormone therapies such as tamoxifen in breast cancer cell lines (Al-Bader et al; 2011.

There are similarly multiple ERβ variants. These also alter the tumor cell response to estradiol in breast cancer cell lines and may be useful in predicting hormone therapy responsiveness to estrogen and anti-estrogen therapies. In particular, cells expressing the ERβ splice variant ERβ2 (or ERβcx) show a poor response to tamoxifen hormone therapy. The ERβ2 variant does not bind ligands and heterodimerizes with ERα, leading to a negative effect on ERα regulated gene expression (Al-Bader et al; 2011).

The nucleosome is the basic unit of chromatin structure and consists of a protein complex of eight highly conserved core histones (comprising of a pair of each of the histones H2A, H2B, H3, and H4). Around this complex is wrapped approximately 146 base pairs of DNA. Another histone, H1 or H5, acts as a linker and is involved in chromatin compaction. The DNA is wound around consecutive nucleosomes in a structure often said to resemble “beads on a string” and this forms the basic structure of open or euchromatin. In compacted or heterochromatin this string is coiled and super coiled into a closed and complex structure (Herranz and Esteller, 2007).

Normal cell turnover in adult humans involves the creation by cell division of some 10¹¹ cells daily and the death of a similar number, mainly by apoptosis. During the process of apoptosis chromatin is broken down into mononucleosomes and oligonucleosomes which are released from the cells into the circulation as cell-free nucleosomes which can be detected by Enzyme-Linked ImmunoSorbant Assay (ELISA). Circulating mononucleosomes contain some 200 base pairs of DNA. Under normal conditions the level of circulating cell-free nucleosomes found in healthy subjects is low. Elevated levels are found in subjects with a variety of conditions including many cancers, auto-immune diseases, inflammatory conditions, stroke and myocardial infarction (Holdenrieder & Stieber, 2009). The measurement of circulating cell free nucleosome adducts has been previously reported (WO 2013/084002).

We now report a rapid, high throughput, non-invasive, low cost, ELISA method for the detection of circulating cell-free nucleosome bound nuclear hormone receptor variant adducts that can be performed with small volumes of blood (10 μl) to detect the progression of hormone sensitive to hormone therapy resistant (or castration resistant) disease and to predict the efficacy of drug treatments for hormone therapy resistant disease in individuals with disease. This method has the additional advantage that it may be repeated as frequently as desired by a simple blood test without the need for biopsy.

### SUMMARY OF THE INVENTION

According to a first aspect of the invention there is provided the use of a cell free nucleosome bound nuclear hormone receptor variant adduct as a biomarker for the progression of hormone dependent cancer to hormone therapy resistant cancer.

According to a second aspect of the invention there is provided the use of a cell free nucleosome bound nuclear hormone receptor variant adduct as a biomarker for the effectiveness of a drug treatment in a patient.

According to a further aspect of the invention there is provided a method of detecting the presence of a cell free nucleosome bound nuclear hormone receptor variant as defined herein isolated from cancer tissue which comprises the steps of chromatin extraction followed by chromatin digestion.

According to a further aspect of the invention there is provided a method of detecting circulating cell free nucleosome bound nuclear hormone receptor variant adduct in a blood, serum or plasma sample taken from a human or animal subject which comprises the steps of:

(i) contacting the sample with a first binding agent which binds to nucleosomes or a component thereof;

(ii) contacting the nucleosomes or sample with a second binding agent which binds specifically to said nuclear hormone receptor variant;

(iii) detecting or quantifying the binding of said second binding agent which binds specifically to said nuclear hormone receptor variant; and

(iv) using the presence or degree of such binding as a measure of the presence of circulating cell free nucleosome bound nuclear hormone receptor variant adducts in the sample.

According to a further aspect of the invention there is provided a method of detecting circulating cell free nucleosome bound nuclear hormone receptor variant adduct in a blood, serum or plasma sample taken from a human or animal subject, which comprises the steps of:

(i) contacting the sample with a first binding agent which binds specifically to said nuclear hormone receptor variant;

(ii) contacting the nucleosomes or sample with a second binding agent which binds to nucleosomes or a component thereof;

(iii) detecting or quantifying the binding of said second binding agent to nucleosomes or a component thereof; and

(iv) using the presence or degree of such binding as a measure of the presence of circulating cell free nucleosome bound nuclear hormone receptor variant adducts in the sample.

### BRIEF DESCRIPTION OF THE FIGURES

FIG. 1: Circulating cell free nucleosome bound AR-V7 ELISA results for 9 cases of treatment naïve newly diagnosed prostate cancer, 1 case of treatment naïve PCA a few months post diagnosis, 1 case of advanced PCA treated by surgery and a negative control (−ve ctrl) sample.

### DETAILED DESCRIPTION OF THE INVENTION

According to a first aspect of the invention there is provided the use of a cell free nucleosome bound nuclear hormone receptor variant adduct as a biomarker for the progression of hormone dependent cancer to hormone therapy resistant cancer.

According to a second aspect of the invention there is provided the use of a cell free nucleosome bound nuclear hormone receptor variant adduct as a biomarker for the effectiveness of a drug treatment in a patient.

In one embodiment, the cell free nucleosome bound nuclear hormone receptor variant adduct comprises a circulating cell free nucleosome bound nuclear hormone receptor variant adduct.

In one embodiment, the circulating cell free nucleosome bound nuclear hormone receptor variant is measured in a blood, serum or plasma sample.

According to a further aspect of the invention there is provided a method of detecting the presence of a cell free nucleosome bound nuclear hormone receptor variant as defined herein isolated from cancer tissue which comprises the steps of chromatin extraction followed by chromatin digestion.

According to a further aspect of the invention there is provided a method of detecting circulating cell free nucleosome bound nuclear hormone receptor variant adduct in a blood, serum or plasma sample taken from a human or animal subject which comprises the steps of:

(i) contacting the sample with a first binding agent which binds to nucleosomes or a component thereof;

(ii) contacting the nucleosomes or sample with a second binding agent which binds specifically to said nuclear hormone receptor variant;

(iii) detecting or quantifying the binding of said second binding agent which binds specifically to said nuclear hormone receptor variant; and

(iv) using the presence or degree of such binding as a measure of the presence of circulating cell free nucleosome bound nuclear hormone receptor variant adducts in the sample.

It will be appreciated that the method of this aspect of the invention is capable of detecting or predicting the progression of a hormone dependent cancer disease to a hormone therapy resistant cancer disease including castration resistant PCA and hormone therapy resistant breast cancer in the subject from whom the sample was taken.

It will also be appreciated that the method of this aspect of the invention is capable of predicting the efficacy of a drug in the subject from whom the sample was taken.

According to a further aspect of the invention there is provided a method of detecting circulating cell free nucleosome bound nuclear hormone receptor variant adduct in a blood, serum or plasma sample taken from a human or animal subject, which comprises the steps of:

(i) contacting the sample with a first binding agent which binds specifically to said nuclear hormone receptor variant;

(ii) contacting the nucleosomes or sample with a second binding agent which binds to nucleosomes or a component thereof;

(iii) detecting or quantifying the binding of said second binding agent to nucleosomes or a component thereof; and

(iv) using the presence or degree of such binding as a measure of the presence of circulating cell free nucleosome bound nuclear hormone receptor variant adducts in the sample.

It will be appreciated that the method of this aspect of the invention is capable of detecting or predicting the progression of a hormone dependent cancer disease to a hormone therapy resistant cancer disease including castration resistant PCA and hormone therapy resistant breast cancer in the subject from whom the sample was taken.

It will also be appreciated that the method of this aspect of the invention is capable of predicting the efficacy of a drug in the subject from whom the sample was taken.

In one embodiment, the hormone receptor variant is a variant of the androgen receptor. In a yet further embodiment, the androgen receptor variant comprises a truncated or short form variant. In a yet further embodiment, the truncated or short form variant of the androgen receptor lacks the ligand binding domain (LBD) of the androgen receptor. In a yet further embodiment, the truncated variant of the androgen receptor comprises androgen receptor variant 7 (AR-V7).

In a further embodiment, the hormone receptor variant is a variant of the androgen receptor and the cancer is selected from castration resistant PCA.

In an alternative embodiment, the hormone receptor variant is a variant of the estrogen receptor. In a yet further embodiment, the estrogen receptor variant comprises a truncated or short form variant. In a yet further embodiment, the truncated or short form variant of the estrogen receptor lacks the ligand binding domain (LBD) of the estrogen receptor. In one embodiment the estrogen receptor variant is ERα-Δ5. In a further embodiment, the estrogen receptor variant is the ERβ splice variant ERβ2 (or ERβcx).

It will be appreciated that the method of this aspect of the invention is capable of detecting or predicting the progression of an estrogen dependent breast cancer disease to a hormone therapy resistant breast cancer disease in the subject from whom the sample was taken.

It will also be appreciated that the method of this aspect of the invention is capable of predicting the efficacy of a drug in the subject from whom the sample was taken.

In a further embodiment, the hormone receptor variant is a variant of the estrogen receptor and the cancer is hormone therapy resistant breast cancer.

In preferred embodiments of the invention a circulating cell free nucleosome bound short form or truncated ER variant adduct is measured in a blood sample taken from a patient. In another preferred embodiment a circulating cell free nucleosome bound short form or truncated AR variant adduct is measured. In both these preferred embodiments the adduct measurements are used to predict the efficacy of a drug for the treatment of the subject from whom the sample as taken and/or to predict or detect the progression of a hormone dependent cancer disease to a hormone therapy resistant cancer disease including castration resistant PCA and hormone therapy resistant breast cancer in the subject from whom the sample was taken.

In particularly preferred embodiments of the invention a circulating cell free nucleosome bound short form or truncated AR-V7 variant adduct is measured in a blood sample taken from a prostate cancer patient and the adduct measurements are used to detect the progression of a hormone dependent prostate cancer disease to a castration resistant prostate cancer.

In further particularly preferred embodiments of the invention a circulating cell free nucleosome bound short form or truncated AR-V7 variant adduct is measured in a blood sample taken from a prostate cancer patient and the adduct measurement is used to predict the efficacy of an androgen agonist or a CYP17 inhibitor for the treatment of the subject from whom the sample was taken. In preferred embodiments of the invention the androgen agonist is enzalutamide. In another preferred embodiment the CYP17 inhibitor is abiraterone.

In a particularly preferred embodiment of the invention, there is provided a method of detecting circulating cell free nucleosome bound AR-V7 adduct in a blood, serum or plasma sample taken from a human or animal subject which comprises the steps of:

(i) contacting the sample with a first binding agent which binds to nucleosomes or a component thereof;

(ii) contacting the nucleosomes or sample with a second binding agent which binds specifically to AR-V7;

(iii) detecting or quantifying the binding of said second binding agent to AR-V7; and

(iv) using the presence or degree of such binding as a measure of the presence of cell-free nucleosome bound AR-V7 adducts in the sample.

It will be appreciated that the method of this embodiment is capable of predicting or detecting the progression of a hormone dependent PCA disease to a castration resistant PCA disease in the subject from whom the sample was taken.

It will also be appreciated that the method of this embodiment is capable of predicting the efficacy of a drug in the subject from whom the sample was taken.

In an alternative particularly preferred embodiment of the invention, there is provided a method of detecting circulating cell free nucleosome bound AR-V7 adduct in a blood, serum or plasma sample taken from a human or animal subject which comprises the steps of:

(i) contacting the sample with a first binding agent which binds specifically to AR-V7;

(ii) contacting the nucleosomes or sample with a second binding agent which binds to nucleosomes or a component thereof;

(iii) detecting or quantifying the binding of said second binding agent to nucleosomes or a component thereof; and

(iv) using the presence or degree of such binding as a measure of the presence of cell-free nucleosome bound AR-V7 adducts in the sample.

It will be appreciated that the method of this embodiment is capable of predicting or detecting the progression of a hormone dependent PCA disease to a castration resistant PCA disease in the subject from whom the sample was taken.

It will also be appreciated that the method of this embodiment is capable of predicting the efficacy of a drug in the subject from whom the sample was taken.

In further embodiments of the invention multiple measurements of different circulating cell free nucleosome bound nuclear hormone receptor variant adducts are performed to ascertain the relative abundance of different variant adducts in a sample, as well as the relative abundance of circulating cell free nucleosome bound wild type nuclear hormone receptor adducts.

A binding agent which binds specifically to a nuclear hormone receptor variant, as used in the present invention, refers to a binding agent which binds strongly and preferentially to a particular variant or isoform, or a particular group of variants or isoforms, but binds less strongly to many, most, or all other variants or isoforms of the nuclear hormone receptor. It will be clear to those skilled in the art that the binding agent used need not bind uniquely to the variant concerned but that this variant should be preferentially bound over other variants commonly present in high levels in samples containing cell free nucleosome nuclear hormone variant adducts. Binding agents which bind specifically to short or truncated variant forms of nuclear hormone receptors are known in the art (Antonarakis).

The binding agents which bind to nucleosomes, as referred to herein, refer to any binding agent which selectively binds to any part of a cell free nucleosome-nuclear hormone receptor variant adduct or complex appropriate to the embodiment. In preferred embodiments, without limitation, the binding agents used are antibodies (including antibody fragments such as light chains or antibody variable regions), aptamers and naturally occurring binding proteins. The binding agent referred to in any of the previous embodiments which binds to nucleosomes or a component thereof, may bind to any component of a nucleosome including without limitation a histone, modified histone, histone variant, DNA or modified DNA component or it may bind to a composite epitope present in intact nucleosomes but not in individual disassociated nucleosome components. In a preferred embodiment of the invention the binding agent referred to in all of the previous embodiments which binds to nucleosomes or a component thereof, is an antibody directed to bind to the histone variant H3.1, H3.2 or H3t.

In further embodiments of the invention any of the methods of the previous embodiments are used but the sample to be tested is a cell free nucleosome preparation derived from cancer tissue removed from a patient (for example by biopsy or surgery). A solution of cell free nucleosomes may be prepared from the tissue by any method known in the art. In a preferred embodiment chromatin is extracted from the cancer tissue and the extract is digested using a nuclease to produce a solution of mononucleosomes. Commercial kits, for example the Pierce Chromatin Prep Module, are available for this purpose.

The invention will now be illustrated with reference to the following non-limiting examples.

### EXAMPLE 1

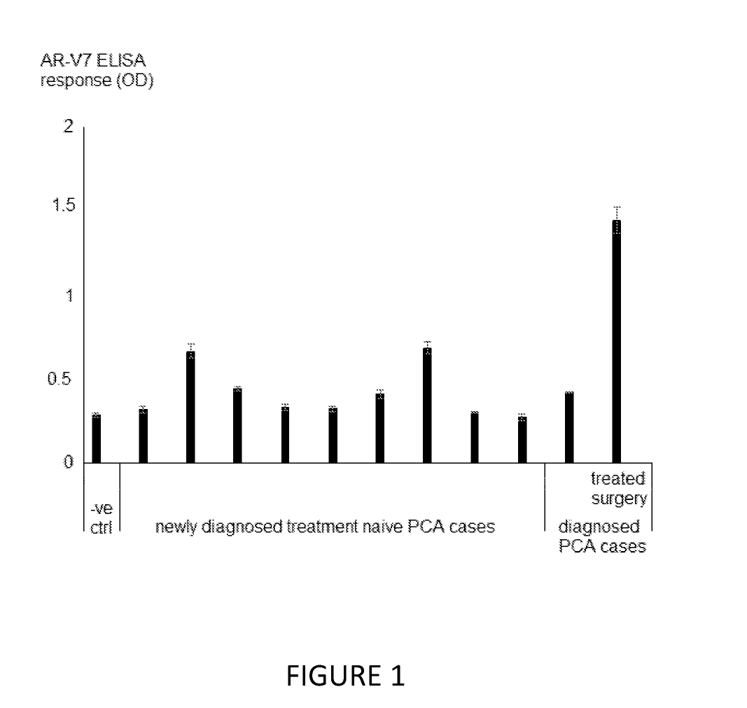

Serum samples were taken from 9 treatment naïve subjects with newly diagnosed prostate cancer and 2 subjects who had been diagnosed within the previous six months. One of these two subjects was also treatment naïve and the other had advanced disease for which surgery was the selected treatment. Blood was drawn from this subject on the day of surgery. A horse serum sample was used as a negative control. The samples were tested for circulating cell free nucleosome bound AR-V7 by an ELISA method using a solid phase anti-histone capture antibody and a biotinylated anti-AR-V7 detection antibody as follows: Serum sample (10 μL/well) and assay buffer (50 μL/well), were added to microtitre plate wells and incubated overnight at 4° C. The serum and assay buffer mixture was decanted and the wells were washed three times with wash buffer. A solution of biotinylated anti-AR-V7 detection antibody was added (50 μL/well) and incubated for 90 minutes at room temperature with mild agitation. Excess detection antibody was decanted and the wells were again washed three times with wash buffer. A solution containing a streptavidin-horse radish peroxidase conjugate was added (50 μL/well) and incubated for 30 minutes at room temperature with mild agitation. Excess conjugate was decanted and the wells were again washed three times with wash buffer. A coloured substrate solution (100 μL/well 2,2′-Azinobis [3-ethylbenzothiazoline-6-sulfonic acid]-diammonium salt) was added and incubated for 20 minutes at room temperature with mild agitation. The optical density (OD) of the wells was measured at a wavelength of 405 nm using a standard microtitre plate reader. A low ELISA signal was observed for all of the 9 treatment naïve newly diagnosed prostate cancer cases as well as the treatment naïve subject that had been diagnosed a few months prior to sampling. These results indicated that no AR variant mediated disease progression, and no development of enzalutamide or abiraterone therapy resistance, had yet occurred in these treatment naïve subjects. However, a high signal was observed for the subject treated with surgery indicating that the disease in this subject had progressed to a castration resistant prostate cancer disease which is also enzalutamide and abiraterone therapy resistant. This data additionally demonstrates that circulating cell free nucleosome nuclear hormone variant adduct levels, measured using the method of the invention, correlate with clinical treatment decisions made by prostate cancer oncologists using current methods for detection of disease progression. The results are shown in FIG. 1.

### EXAMPLE 2

Serum samples were taken from treatment naïve subjects with newly diagnosed breast cancer and from subjects with treated breast cancer. The samples were tested for circulating cell free nucleosome bound ERα-Δ5 by an ELISA method similar to that described in EXAMPLE 1 above but instead using a biotinylated anti-ERα-Δ5 detection antibody. Low ELISA signals were observed in the treatment naïve newly diagnosed breast cancer cases. The results indicated that there had been no development of ERα-Δ5 mediated hormone therapy resistance in these treatment naïve subjects. However, a high signal was observed for some of the subjects with treated breast cancer disease indicating elevated levels of circulating cell free nucleosome bound ERα-Δ5 variant adducts in the serum samples from those subjects and disease progression to a hormone therapy resistant breast cancer disease in those subjects.

### EXAMPLE 3

Serum samples were taken from treatment naïve subjects with newly diagnosed breast cancer and from subjects with treated breast cancer. The samples were tested for circulating cell free nucleosome bound ERβ2 by an ELISA method similar to that described in EXAMPLE 1 above but instead using a biotinylated anti-ERβ2 detection antibody. Low ELISA signals were observed in the treatment naïve newly diagnosed breast cancer cases. The results indicated that there had been no development of ERβ2 mediated hormone therapy resistance in these treatment naïve subjects. However, a high signal was observed for some of the subjects with treated breast cancer disease indicating elevated levels of circulating cell free nucleosome bound ERβ2 variant adducts in the serum samples from those subjects and disease progression to a hormone therapy resistant breast cancer disease in those subjects.

### EXAMPLE 4

A cancer tissue biopsy sample was taken from treatment naïve subjects with newly diagnosed prostate cancer and from subjects with treated prostate cancer. A solution of cell free mononucleosomes and/or oligonucleosomes was prepared from the tissue samples using the Pierce Chromatin Prep Module according to the manufacturer's instructions. The samples were tested for cell free nucleosome bound AR-V2 by an ELISA method similar to that described in EXAMPLE 1 above. Low ELISA signals were observed in the treatment naïve newly diagnosed prostate cancer cases. The results indicated that there had been no development of AR-V7 mediated hormone therapy resistance in these treatment naïve subjects. However, a high signal was observed for some of the subjects with treated prostate cancer disease indicating elevated levels of cell free nucleosome bound AR-V7 variant adducts in the samples from those subjects and disease progression to a hormone therapy resistant prostate cancer disease in those subjects which is also enzalutamide and abiraterone therapy resistant.

### EXAMPLE 5

A cancer tissue biopsy sample was taken from treatment naïve subjects with newly diagnosed breast cancer and from subjects with treated breast cancer. A solution of cell free mononucleosomes and/or oligonucleosomes was prepared from the tissue samples using the Pierce Chromatin Prep Module according to the manufacturer's instructions. The samples were tested for cell free nucleosome bound ERα-Δ5 by an ELISA method similar to that described in EXAMPLE 2 above. Low ELISA signals were observed in the treatment naïve newly diagnosed breast cancer cases. The results indicated that there had been no development of ERα-Δ5 mediated hormone therapy resistance in these treatment naïve subjects. However, a high signal was observed for some of the subjects with treated breast cancer disease indicating elevated levels of cell free nucleosome bound ERα-Δ5 variant adducts in the samples from those subjects and disease progression to a hormone therapy resistant breast cancer disease in those subjects.

### EXAMPLE 6

A cancer tissue biopsy sample was taken from treatment naïve subjects with newly diagnosed breast cancer and from subjects with treated breast cancer. A solution of cell free mononucleosomes and/or oligonucleosomes was prepared from the tissue samples using the Pierce Chromatin Prep Module according to the manufacturer's instructions. The samples were tested for cell free nucleosome bound ERβ2 by an ELISA method similar to that described in EXAMPLE 3 above. Low ELISA signals were observed in the treatment naïve newly diagnosed breast cancer cases. The results indicated that there had been no development of ERβ2 mediated hormone therapy resistance in these treatment naïve subjects. However, a high signal was observed for some of the subjects with treated breast cancer disease indicating elevated levels of cell free nucleosome bound ERβ2 variant adducts in the samples from those subjects and disease progression to a hormone therapy resistant breast cancer disease in those subjects.

### REFERENCES

Al-Bader et al. (2011) Experimental and therapeutic medicine; 2, 537-44.

Antonarakis et al. (2014) N Engl J Med; 371:1028-38.

Dehm et al. (2008) Cancer Res; 68(13), 5469-77, 2008.

Fuqua et al. (1999) Cancer Res; 59, 5425-28, 1827-33.

Huang et al. (2007) American J Pathology; 150(5), 1997

Herranz and Esteller (2007) Methods Mol Biol. 361:25-62.

Holdenrieder and Stieber (2009) Critical Reviews in Clinical Laboratory Sciences; 46(1): 1-24, 2009.

1-4. (canceled)

5. A method of detecting the presence of a cell free nucleosome bound nuclear hormone receptor variant isolated from cancer tissue which comprises the steps of chromatin extraction followed by chromatin digestion.

6. A method of detecting circulating cell free nucleosome bound nuclear hormone receptor variant adduct in a blood, serum or plasma sample taken from a human or animal subject which comprises the steps of:
(i) contacting the sample with a first binding agent which binds to nucleosomes or a component thereof;
(ii) contacting the nucleosomes or sample with a second binding agent which binds specifically to said nuclear hormone receptor variant;
(iii) detecting or quantifying the binding of said second binding agent which binds specifically to said nuclear hormone receptor variant; and
(iv) using the presence or degree of such binding as a measure of the presence of circulating cell free nucleosome bound nuclear hormone receptor variant adducts in the sample.

7. A method of detecting circulating cell free nucleosome bound nuclear hormone receptor variant adduct in a blood, serum or plasma sample taken from a human or animal subject, which comprises the steps of:
(i) contacting the sample with a first binding agent which binds specifically to said nuclear hormone receptor variant;
(ii) contacting the nucleosomes or sample with a second binding agent which binds to nucleosomes or a component thereof;
(iii) detecting or quantifying the binding of said second binding agent to nucleosomes or a component thereof; and
(iv) using the presence or degree of such binding as a measure of the presence of circulating cell free nucleosome bound nuclear hormone receptor variant adducts in the sample.

8. The method as defined in any one of claims 5 to 7, wherein the hormone receptor variant is a variant of the androgen receptor, such as a truncated or short form variant.

9. The method of claim 8, wherein the truncated or short form variant of the androgen receptor lacks the ligand binding domain (LBD) of the androgen receptor.

10. The method of claim 8, wherein the truncated variant of the androgen receptor comprises androgen receptor variant 7 (AR-V7).

11. The method as defined in any one of claims 5 to 7, wherein the hormone receptor variant is a variant of the androgen receptor and the cancer is selected from castration resistant PCA.

12. The method as defined in any one of claims 5 to 7, wherein the hormone receptor variant is a variant of the estrogen receptor, such as a truncated or short form variant.

13. The method as defined in claim 12, wherein the truncated or short form variant of the estrogen receptor lacks the ligand binding domain (LBD) of the estrogen receptor.

14. The method as defined in claim 12, wherein the estrogen receptor variant is ERα-Δ5.

15. The method as defined in claim 12, wherein the estrogen receptor variant is the ERβ splice variant ERβ2 (or Eβcx).

16. The method as defined in any one of claims 5 to 7, wherein the hormone receptor variant is a variant of the estrogen receptor and the cancer is hormone therapy resistant breast cancer.

17. The method as defined in claim 10, wherein the AR-V7 variant adduct is measured in a blood sample taken from a prostate cancer patient and the adduct measurement is used to predict the efficacy of an androgen agonist, such as enzalutamide, or a CYP17 inhibitor, such as abiraterone, for the treatment of the subject from whom the sample was taken.

18. A method of detecting circulating cell free nucleosome bound AR-V7 adduct in a blood, serum or plasma sample taken from a human or animal subject which comprises the steps of:
(i) contacting the sample with a first binding agent which binds to nucleosomes or a component thereof;
(ii) contacting the nucleosomes or sample with a second binding agent which binds specifically to AR-V7;
(iii) detecting or quantifying the binding of said second binding agent to AR-V7; and
(iv) using the presence or degree of such binding as a measure of the presence of cell-free nucleosome bound AR-V7 adducts in the sample.

19. A method of detecting circulating cell free nucleosome bound AR-V7 adduct in a blood, serum or plasma sample taken from a human or animal subject which comprises the steps of:
(i) contacting the sample with a first binding agent which binds specifically to AR-V7;
(ii) contacting the nucleosomes or sample with a second binding agent which binds to nucleosomes or a component thereof;
(iii) detecting or quantifying the binding of said second binding agent to nucleosomes or a component thereof; and
(iv) using the presence or degree of such binding as a measure of the presence of cell-free nucleosome bound AR-V7 adducts in the sample.
